(19) United States
(12) Patent Application Publication
(10) Pub. No.: US 2015/0088090 A1
(43) Pub. Date: Mar. 26, 2015

(54) METHOD AND APPARATUS FOR A CLOG RESISTANT ORIFICE

(71) Applicant: HOSPI CORPORATION, Concord, CA (US)

(72) Inventor: Bradford MACY, JR., Concord, CA (US)

(21) Appl. No.: 14/463,897

(22) Filed: Aug. 20, 2014

Related U.S. Application Data
(63) Continuation of application No. PCT/US2013/026951, filed on Feb. 20, 2013.
(60) Provisional application No. 61/633,999, filed on Feb. 21, 2012.

(51) Int. Cl. A61M 25/00; A61M 1/00

(52) U.S. Cl. CPC A61M 25/007; A61M 25/0054; A61M 1/0078
USPC 604/500; 604/266

(57) ABSTRACT

Devices, apparatuses, systems, and methods for improved clog resistant orifices in medical devices and tubes are provided. A medical tube will comprise a tubular wall having one or more clog resistant orifices. The clog resistant orifices have an outwardly flared orifice wall so as to minimize the occurrence of clogging. The outward flaring, and in some cases inward tapering relative to the outer surface of the tubular wall, can minimize tension and compression forces from forming within the orifice. These forces can otherwise maintain a clog in position within the orifice. The portions of the tubular wall adjacent the orifices may also be more flexible than the remainder of the tubular wall, allowing such portions to bend under pressure or suction to facilitate the shearing away of any clog from the orifice.

### CROSS-REFERENCE

This application is a continuation of PCT Application No. PCT/US2013/026951, filed Feb. 20, 2013, which claims the benefit of U.S. Provisional Application No. 61/633,999, filed Feb. 21, 2012, the full contents of which are incorporated herein by reference.

### BACKGROUND OF THE INVENTION

The present disclosure relates to medical devices, systems, and methods. In particular, improved medical devices, systems, and methods are provided to improve resistance against the clogging of the various ports or orifices in the various medical devices.

Medical tubes used for removal or delivery of a substance into or out of a body cavity typically have some form of transmission holes near the end of the tube, which transmit the particular substance into or out of the body cavity. Many tubes used such purposes clog frequently. Body secretions, bacterial biofilm, or accumulating residue can tend to build up around these holes or orifices causing clogging and subsequent malfunction of the device. Theoretically, the larger the hole the less it is able to clog. But, orifice size for many medical devices is often limited depending on the size of the tube and its utility. In order to create a clog resistant orifice, other design components may need to be taken into account. An orifice design which can exploit both the physics of the forces acting on a clogged orifice, and on the tube structure immediately surrounding the orifice may be highly desirable. The designs of existing transmission holes on medical tubes often do not take into account these concerns. Thus, clogs within medical tubes are all too frequent.

FIG. 1A is an illustration of transmission orifices or holes typically present in many current medical tubes or other devices. The transmission orifices or holes are cut perpendicular to the surface of the lumen and outer wall of the tube. FIG. 1A shows the tip of a typical tube with several orifices or holes 01 or 02 in the tube wall 04. In FIG. 1A, a hole 01 is clogged. Fluid is being pressured into the lumen to irrigate the tube as shown by arrows 03, and the fluid is going out the open holes 02, and applying a pressure to the clogged hole 01. FIG. 1B shows an enlarged view of the hole 01. Fluid pressure is applying a force, represented by pressure force arrow 07, to the clog in the direction indicated by the arrow 07. Fluid cannot pass through the hole 01 from the inner lumen surface 06 to the outer tube surface 05 due to the clog. FIG. 1B demonstrates the different forces at work within a clog 09, the orifice 01, and the wall of the tubing 04 which defines the orifice. Each of the various arrows represents force vectors. For reference, these forces are (i) the pressure force 07 exerted by the irrigation fluid, (ii) the adhesive force 08 between the wall and the clog, (iii) the tension force 10 from compaction of the clogged substance into the hole, the compression force 12 from the stretching of the tube wall and the retraction against the clog, and (iv) the cohesive force 11 which is the interaction between like particles of the clogged substance which forms the clog.

As shown in FIG. 1B, the device is clogged, and attempts are made to clear the clog by irrigating the tube with a fluid. The force of the fluid attempts to push the clog out by applying a pressure force in the direction of the clog 07. The clog is held together primarily by cohesive forces 11 of the particles making up the clog. These particles may be stool, mucous, biofilm, etc. Along the wall of the clogged hole, adhesive forces 08 of the substance to the wall work against the force of the fluid through a shearing force between the wall and the clog. In addition, if the clog has been under any type of pressure, tension forces 10 of the clog against the wall also work against the fluid force 07. An example of the creation of tension forces would be in the rectum where stool can be compressed into the holes of a tube by forces of the rectal muscle, creating an orifice which is tightly packed with stool.

Lastly, if the tube is flexible, forces of compression from the tube against the clog also come into play. More simply stated, the clog becomes packed into the hole with only a small open area in which the force of fluid can act against the forces holding the clog in place. These tension and compression forces can be much larger than the adhesive forces holding the clog to the wall, or the cohesive forces forming the clog in the first place. They can easily make the difference between being able to clear a clog with an irrigation fluid and being unable to clear the clog. An orifice that does not allow for the buildup of compression and tension forces would be highly superior to currently known and used transmission holes.

### SUMMARY OF THE INVENTION

Devices, apparatuses, systems, and methods are provided for a clog resistant orifice which exploits both the physics of the forces acting within a clogged orifice and the surrounding tube structure. The non-clogging or clog resistant orifices or holes according to the present disclosure minimize the occurrence of clogging and reduce the ability of clogs to block an orifice by one or more of the following: (i) eliminating tension and compression forces from forming within the hole, (ii) allowing the inner hole diameter to expand, which (a) can increase the pressure force exerted on the clog exponentially where P≈d² and (b) can cause shearing of the clog away from the wall when expansion occurs, and (iii) causing the wall to bend outward, away from the lumen when pressure is applied, or inward toward the lumen when suction is applied, which causes shearing of the clog away from the wall when this bending occurs. While the devices, apparatuses, systems, and methods disclosed herein find particular application for medical tubes, they may be useful and applied for other forms of tubing as well.

A first aspect of the disclosure provides a tube, typically a medical tube, having reduced occurrence of clogging. The tube comprises a tubular wall having one or more clog resistant orifices. The tube will typically be flexible. The clog resistant orifices have an outwardly flared orifice wall so as to minimize the occurrence of clogging. The orifice wall may be flared outwardly at an angle of greater than or equal to 90 degrees, 110 degrees, 130 degrees, 160 degrees, or even 170 degrees relative to the inner surface of the tubular wall. In some cases, the orifice wall may have an inner portion outwardly flared at a first angle of greater than 90 degrees relative to the inner surface of the tubular wall and an outer portion inwardly tapered at a different second angle of greater than 90 degrees relative to the outer surface of the tubular wall. For example, the first angle may be greater than or equal to 160 degrees and the second angle may be greater than or equal to 130 degrees. Typically, the clog resistant orifices will comprise an inner opening adjacent the inner surface of the tubular wall and an outer opening adjacent the outer surface of the tubular wall, with the outer opening being larger than the inner opening. The outwardly flaring or inwardly tapering shapes of the orifices can minimize tension and compression forces from forming within the orifice.

The portions of the tubular wall adjacent the orifices may be more flexible than the remainder of the tubular wall. These portions may deform, for example by bending outwardly, when under fluid pressure from within the tube or bend inwardly when exposed to suction from within the tube. The one or more portions of the tubular wall adjacent the one or more orifices may be thinner than the remainder of the tubular wall. These structural properties can allow the inner hole diameter to expand or allow the wall to bend, facilitating the shearing of any clog away from the orifice wall.

Another aspect of the disclosure provides a method of reducing the occurrence of clogging in a tube. The tube having one or more clog resistant orifices described above is provided.

Yet another aspect of the disclosure provides another method of reducing the occurrence of clogging in a tube, typically a medical tube. One or more clog resistant orifices are formed in the tube such than an orifice wall of the orifices is outwardly flared so as to minimize the occurrence of clogging. The orifice wall may be formed such that the wall flares outwardly at an angle of greater than or equal to 90 degrees, 110 degrees, 130 degrees, 160 degrees, or even 170 degrees. In some cases, the orifice wall may be formed to have an inner portion outwardly flared at a first angle of greater than 90 degrees relative to the inner surface of the tubular wall and an outer portion inwardly tapered at a different second angle of greater than 90 degrees relative to the outer surface of the tubular wall. For example, the first angle may be greater than or equal to 160 degrees and the second angle may be greater than or equal to 130 degrees. Typically, the clog resistant orifices will be formed so as to comprise an inner opening adjacent the inner surface of the tubular wall and an outer opening adjacent the outer surface of the tubular wall, with the outer opening being larger than the inner opening. Again, the outwardly flaring or inwardly tapering shapes of the orifices can minimize tension and compression forces from forming within the hole.

The orifices may be formed such that the portions of the tubular wall adjacent the orifices are more flexible than the remainder of the tubular wall. These portions may deform, for example by bending outwardly, when under fluid pressure from within the medical tube or bending inwardly when exposed to suction from within the medical tube. The orifices may also be formed such that one or more portions of the tubular wall adjacent the orifices may be thinner than the remainder of the tubular wall. Again, these structural properties can allow the inner hole diameter to expand or allow the wall to bend, facilitating the shearing of any clog away from the orifice wall. The material forming the walls of a non-clogging orifice may be made of any manner of soft, pliable materials. Additionally or alternatively, these walls may have surface treatments or coatings, such as fluorinated compounds or various plasma treatments, which reduce adhesion forces of a given surface. Furthermore, microsurfaces, for example as in lotus leaves or artificial versions of microprotrusions, may also be provided on the outer surfaces of the orifice walls to reduce effective wetting angle and surface forces.

A further aspect of the disclosure provides a method of removing a clog from an orifice in a tube, typically a medical tube. Fluid pressure or suction is applied from within the tube to deform a portion of the tubular wall of the tube adjacent the orifice so as to push the clog away from the orifice. Fluid pressure applied from within the tube may deform this portion of the tubular wall by bending said portion outwardly. Suction applied from within the tube may deform this portion of the tubular wall by bending said portion inwardly. Typically, the orifice will comprise an inner opening adjacent the inner surface of the tubular wall and an outer opening adjacent an outer surface of the tubular wall, with the outer opening being larger than the inner opening.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a longitudinal section of a currently known and used medical tube with currently known and used transmission orifices or holes;

FIG. 1B shows a magnified view of one of the clogged transmission orifices or holes in the medical tube of FIG. 1A, including the forces acting on the clog, orifice, and surrounding wall;

FIG. 2A shows a longitudinal section of a medical tube with clog resistant orifices according to embodiments of the present disclosure;

FIG. 2B shows a magnified view of an un-clogged transmission orifice of the medical tube of FIG. 2A;

FIG. 2C shows a magnified view of a nearly clogged transmission orifice of the medical tube of FIG. 2A, including the forces acting on the potential clog, orifice, and surrounding wall;

FIGS. 3A to 3C show a magnified view of a nearly clogged transmission orifice of a medical tube according to embodiments of the present disclosure as pressure is applied to the clog from fluid within the tube;

FIG. 4A show an outside view of the outer surface of the medical tube with a clog resistant orifice, including the accumulation of a clog around the inner hole, according to embodiments of the disclosure;

FIG. 4B show an outside view of the outer surface of the medical tube with a clog resistant orifice, including how the diameter of the inner hole expands when pressure is exerted, stretching the surface and causing shearing of the clog from the wall of the orifice, according to embodiments of the disclosure;

FIG. 5A shows a magnified view of a clog resistant transmission orifice of a medical tube according to further embodiments of the present disclosure;

FIG. 5B shows a magnified view of the clog resistant transmission orifice of FIG. 5A with the inner wall bending outward when pressure is applied; and

FIG. 5C shows a magnified view of the clog resistant transmission orifice of FIG. 5A with the inner wall bending inward when pressure is applied.

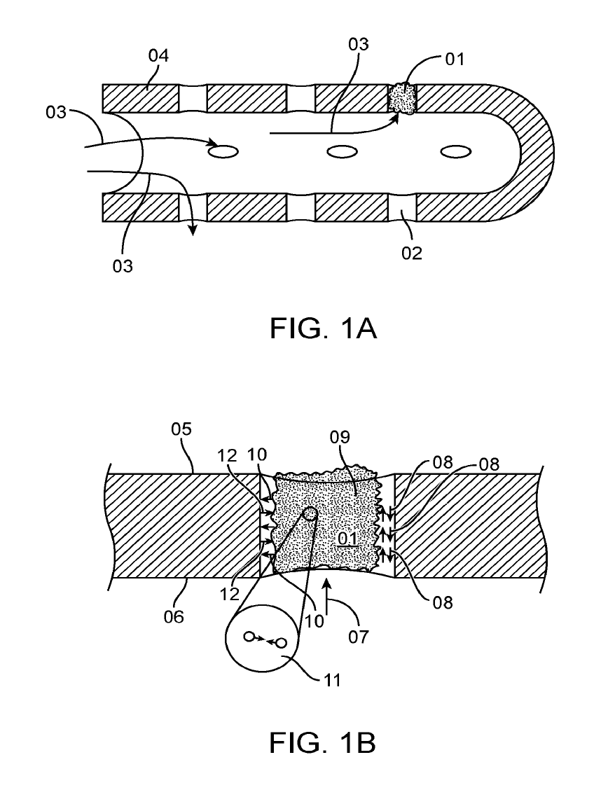

### DETAILED DESCRIPTION OF THE INVENTION

Devices, apparatuses, systems, and methods are provided for a clog resistant orifice which exploits both the physics of the forces acting within a clogged orifice and the surrounding tube structure.

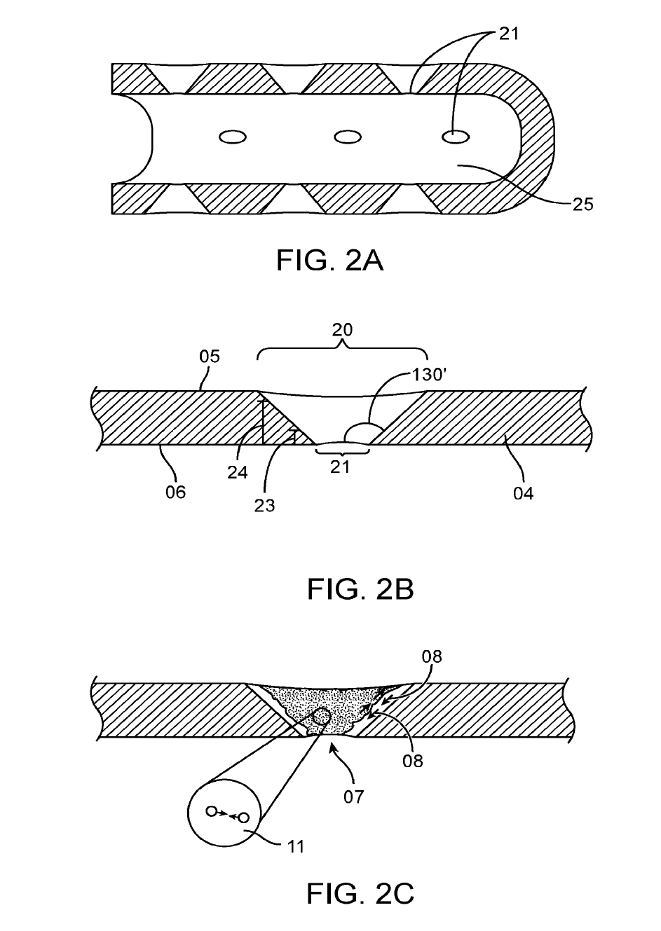

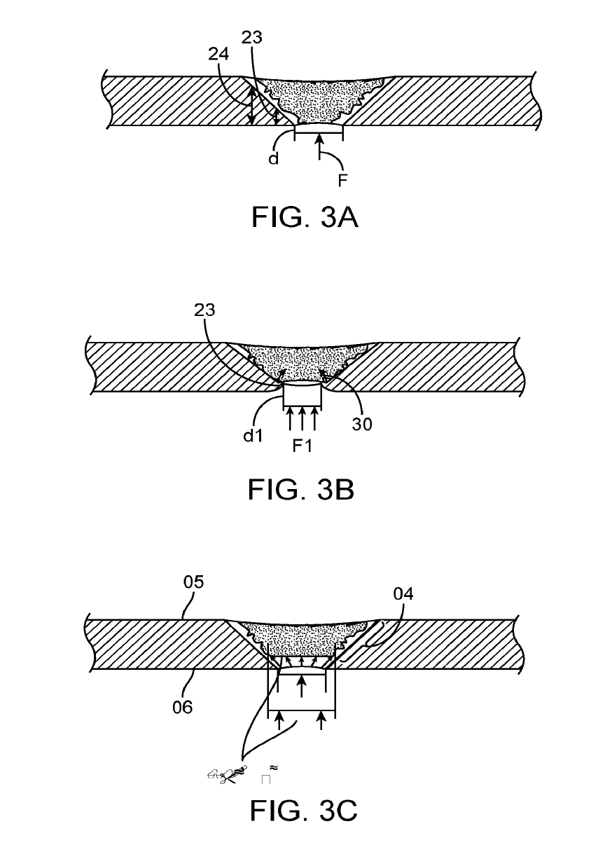

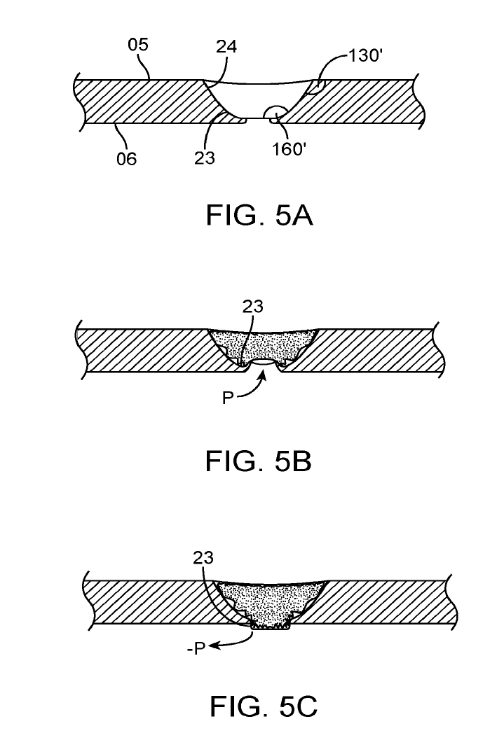

FIGS. 2A to 2B are illustrations of one embodiment of a clog resistant orifice. In this embodiment, a clog resistant orifice has an inner opening or hole 21 which opens into the lumen 25 of the device or tube. The inner hole is in fluid communication with an outer opening or hole 20 which opens into the outside of the tube, which, in some embodiments, would be in contact with a body cavity for which the tube is either instilling or draining a substance. In the present embodiment, the wall of the tube 04 is cut in a circular or oval shape with the outer wall 24 cut in a larger diameter than the inner wall 23 such that the angle of the wall forming the orifice is angled at greater than 90° in relation to the lumen surface 06 and outer tube surface 05. In the embodiment of the present invention the angle is 130°, but this angle could vary from 110° to 170° within different embodiments. The angle within any embodiment would be sufficient to allow the inner wall to be thin enough to bend significantly outward (as with the clog resistant orifices shown, for example, by FIGS. 3B and 5B described below) when pressure or is applied from the inner lumen, and bend inward significantly (as with the clog resistant orifice shown, for example, by FIG. 5C described below) when suction is applied from the inner lumen.

FIG. 2A shows the end of a tube with several non-clogging holes. FIG. 2B shows a close-up view of a clog resistant orifice which has formed a clog, including the various forces described above which may act on the clog. In this embodiment, the improved clog resistant orifice is shaped at an angle of 130° in relation to the inner wall 08, with the inner opening or hole 21 being much smaller than the outer opening or hole 20. The angle of the cut can serve several functions. First, since the wall of the hole is angled substantially greater than 90°, no tension or compression forces can occur which are greater than the adhesive force or cohesive forces, leaving only the adhesive forces 08 and cohesive forces 11 to overcome in order to clear the clog from the orifice. As shown in FIG. 2C, the only forces acting to hold the clog in the hole are the adhesive forces between the wall and the clog and the cohesive forces holding the clog together. Secondly, since the thickness of the inner wall 23 within this embodiment is much thinner than the thickness at the outer wall 24, the inner wall 23 bends outward when pressure is exerted by the fluid, and inward into the lumen when suction force is applied, which assists in forcing the clog off the wall by shearing action. The bending of the inner wall can also serve to enlarge the inner holes diameter, which increases the force applied to the clog as is illustrated in FIGS. 3A to 3C.

FIGS. 3A to 3C illustrate a method of clearing a clogged orifice whereby pressure or suction is applied to the inner lumen of a tube with clog resistant orifices. In FIG. 3A, the inner hole has a diameter d when no pressure is applied to the lumen of the tube. In FIG. 3B, a pressure force F on the inner hole bends the inner wall 23 outward and stretches the inner hole, making the making the diameter d₁ larger. This allows for a larger surface area for the pressure force F₁ to act on the clog. This larger surface area increases the force applied to the clog exponentially, where F≈d², and F₁≈d₁².

FIG. 3A shows a pressure force F being applied by a fluid on the clog. The force is applied is proportional to the square of the diameter d of the hole at the lumen. When the fluid force is applied, the inner wall bends 23 in the direction of the clog, and this bending outward increases the diameter of the hole D1, which increases the force F1 applied to the clog exponentially as shown in FIGS. 3B and 3C. In addition, the adhesive forces holding the clog in place adjacent to the opening of the hole at the inner lumen may be exposed to increased shearing forces 30 from the bending of the tube wall and the shearing forces thus created. These forces act to tear the clog away from the wall, which expose still more area for the pressure force to work against the adhesive and cohesive forces of the clog. FIG. 3C shows how the clog has been broken away from the wall at the area immediately adjacent to the inner lumen and now the fluid can enter the hole further in, working on a larger area of the cohesive and adhesive forces of the clog.

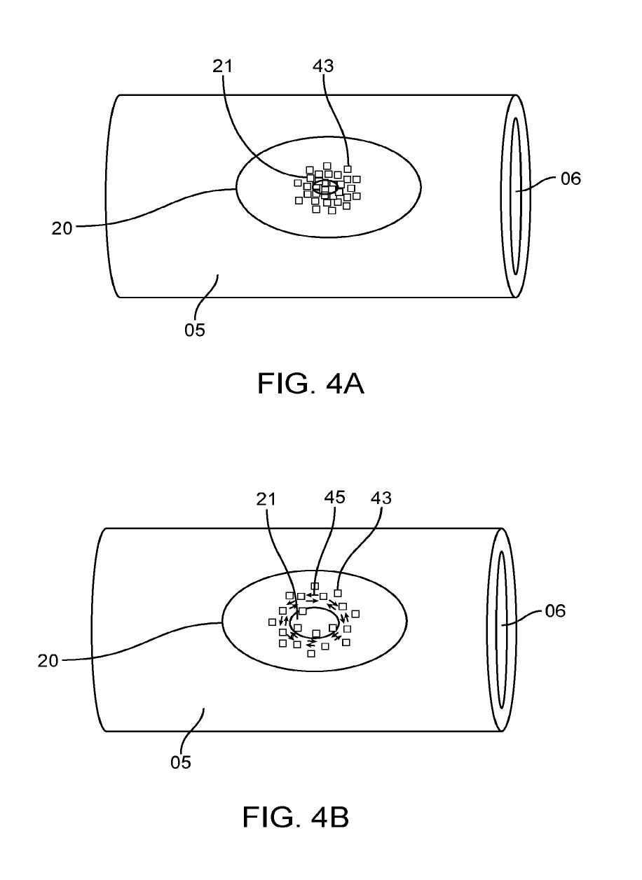

FIGS. 4A and 4B show an enlarged outside view of the outer wall surface 05 of a medical tube with a clog resistant orifice. FIG. 4A shows how clogging particles 43 have formed around the inner hole 21 of the orifice. FIG. 4B shows the same tubing segment with pressure applied to the inner lumen. FIG. 4B illustrates how the increased diameter of the inner hole 21 created when fluid pressure is applied can cause a shearing 45 of the wall immediately surrounding the orifice by a stretching action when the inner hole expands. This stretching of the flexible material that makes up the wall of the orifice tears the particles from the wall and re-opens the orifice.

As illustrated in FIG. 4A and FIG. 4B, methods disclosed herein further employ a shearing force 45, created by the stretching and bending of the inner wall which acts to tear the clog from the wall. FIG. 4A illustrates how particles 43 accumulate to form a clog on and around the inner hole of a clog resistant orifice. In FIG. 4a, the inner hole 21 is completely clogged. In FIG. 4B, pressure is applied to the inner lumen and the inner hole 21 stretches shearing away the particles and clearing the clog.

Lastly, the methods disclosed herein employ the an angle of the orifice whereby the inner hole is smaller than the outer hole, and the wall is cut at an angle, which is substantially greater than 90° in relation to the inner lumen and outer tube wall. This angle serves to inhibit the formation of any substantial compression or tension forces within the hole.

FIGS. 5A to 5C are illustrations of an alternative embodiment of the present invention, whereby the thinness of the inner wall is maximized by increasing the angle at the inner wall 23 in relation to the outer wall 24 angle. FIG. 5A shows a magnified view of a clog resistant transmission orifice of a medical tube according to further embodiments of the present disclosure, FIG. 5B shows a magnified view of the clog resistant transmission orifice with the inner wall bending outward when pressure is applied, and FIG. 5C shows a magnified view of the clog resistant transmission orifice of FIG. 5C with the inner wall bending inward when pressure is applied. In the embodiment of FIGS. 5A to 5C, the angle close to the outer wall is 130°, and the angle close to the inner wall is 160°, but these angles may vary in different embodiments. One utility of this embodiment may be to maximize the angle of the inner hole while minimizing the size of the outer hole.

It will be apparent to a skilled artisan that the embodiments described herein are exemplary of inventions that may have greater scope than any of the singular descriptions presented. There may be alterations made in these examples without departing from the spirit and scope of the method and apparatus of the invention disclosed. For example, any portion of a clog resistant orifice may have varying shapes, angles, or designs within different embodiments while still employing the method, and achieving the purpose of a clog resistant orifice as described in the present disclosure. The walls of invention may be made of various materials or have any manner of coatings or treatments without departing from the spirit and scope of the method and apparatus of the invention disclosed.

1. A medical tube having reduced occurrence of clogging, the medical tube comprising:
a tubular wall having one or more clog resistant orifices, wherein the one or more clog resistant orifices have an outwardly flared orifice wall so as to minimize the occurrence of clogging for the one or more clog resistant orifices.

2. The medical tube of claim 1, wherein the tubular wall has an inner surface and an outer surface, and wherein the one or more clog resistant orifices comprises an inner opening adjacent the inner surface of the tubular wall and an outer opening adjacent the outer surface of the tubular wall, the outer opening being larger than the inner opening.

3. The medical tube of claim 1, wherein the tube is flexible.

4. The medical tube of claim 1, wherein one or more portions of the tubular wall adjacent the one or more orifices are more flexible than the remainder of the tubular wall.

5. The medical tube of claim 4, wherein the one or more portions of the tubular wall adjacent the one or more orifices is thinner than the remainder of the tubular wall.

6. The medical tube of claim 1, wherein one or more portions of the tubular wall adjacent the one or more orifices are configured to deform when under fluid pressure from within the tube.

7. The medical tube of claim 6, wherein the one or more portions of the tubular wall adjacent the one or more orifices are configured to bend outwardly when under fluid pressure from within the tube.

8. The medical tube of claim 1, wherein one or more portions of the tubular wall adjacent the one or more orifices are configured to deform when under suction from within the tube.

9. The medical tube of claim 8, wherein the one or more portions of the tubular wall adjacent the one or more orifices are configured to bend inwardly when exposed to suction from within the medical tube.

10. The medical tube of claim 1, wherein the tubular wall has an inner surface and the orifice wall is flared outwardly at an angle of greater than or equal to 90 degrees relative to the inner surface of the tubular wall.

11. The medical tube of claim 10, wherein the orifice wall is flared outwardly at an angle of greater than or equal to 110 degrees relative to the inner surface of the tubular wall.

12. The medical tube of claim 11, wherein the orifice wall is flared outwardly at an angle of greater than or equal to 130 degrees relative to the inner surface of the tubular wall.

13-14. (canceled)

15. The medical tube of claim 1, wherein the tubular wall has an inner surface and an outer surface, and wherein the orifice wall of the one or more clog resistant orifices comprises an inner portion outwardly flared a first angle of greater than 90 degrees relative to the inner surface of the tubular wall and an outer portion inwardly tapered at a second angle of greater than 90 degrees relative to the outer surface of the tubular wall.

16. The medical tube of claim 15, wherein the first angle is greater than or equal to 160 degrees and the second angle is greater than or equal to 130.

17. A method of reducing the occurrence of clogging in a medical tube, the method comprising providing the medical tube of claim 1.

18. A method of reducing the occurrence of clogging in a medical tube, the method comprising:
forming one or more clog resistant orifices in the medical tube such than an orifice wall of the one or more orifices is outwardly flared so as to minimize the occurrence of clogging for the one or more clog resistant orifices.

19. The method of claim 18, wherein the one or more clog resistant orifices are formed such that the one or more orifices comprise an inner opening adjacent the inner surface of the tubular wall and an outer opening adjacent an outer surface of the tubular wall, the outer opening being larger than the inner opening.

20-21. (canceled)

22. The method of claim 18, wherein the one or more clog resistant orifices are formed such that one or more portions of the tubular wall adjacent the one or more clog resistant orifices are more flexible than the remainder of the tubular wall.

23-26. (canceled)

23. The method of claim 18, wherein the one or more clog resistant orifices are formed such that the orifice wall is outwardly flared at an angle of greater than or equal to 90 degrees relative to an inner surface of the tubular wall.

28-31. (canceled)

32. The method of claim 18, wherein the tubular wall has an inner surface and an outer surface, and the one or more clog resistant orifices are formed such that the orifice wall thereof comprises an inner portion flared outwardly at a first angle of greater than 90 degrees relative to the inner surface of the tubular wall and an outer portion tapered inwardly at a second angle of greater than 90 degrees relative to the outer surface of the tubular wall.

33. (canceled)

34. A method of removing a clog from an orifice in a medical tube, the method comprising:
applying fluid pressure or suction from within the tube to deform a portion of a tubular wall of the tube adjacent the orifice so as to push the clog away from the orifice.

35. The method of claim 34, wherein fluid pressure is applied from within the tube to deform the portion of the tubular wall by bending said portion outwardly.

36. The method of claim 34, wherein suction is applied from within the tube to deform the portion of the tubular wall by bending said portion inwardly.

37. The method of claim 34, wherein the orifice comprises a orifice wall that is flared outwardly relative to an inner surface of the tubular wall.

38. The method of claim 37, wherein the orifice comprises an inner opening adjacent the inner surface of the tubular wall and an outer opening adjacent an outer surface of the tubular wall, the outer opening being larger than the inner opening.
